(19) United States
(12) Patent Application Publication
(10) Pub. No.: US 2022/0354756 A1
(43) Pub. Date: Nov. 10, 2022

(54) A WATER-IN-OIL EMULSION WITH EMOLLIENT AND METHODS OF USING THE SAME

(71) Applicant: SHISEIDO COMPANY, LTD., Tokyo (JP)

(72) Inventors: Lethu NGUYEN, East Windsor, NJ (US); Hinaben PATEL, East Windsor, NJ (US)

(73) Assignee: SHISEIDO COMPANY, LTD., Tokyo (JP)

(21) Appl. No.: 17/624,151

(22) PCT Filed: Jul. 22, 2020
(86) PCT No.: PCT/IB2020/056889

§ 371 Date: Dec. 30, 2021

Related U.S. Application Data
(60) Provisional application No. 62/878,059, filed on Jul. 24, 2019.

(51) Int. Cl. A61K 8/06; A61K 8/04; A61K 8/81; A61K 8/41; A61K 8/63; A61Q 1/04

(52) U.S. Cl. CPC A61K 8/064; A61K 8/042; A61K 8/8117; A61K 8/416; A61K 8/63; A61Q 1/04; A61K 2800/92

(57) ABSTRACT

The present disclosure relates generally to compositions that include at least two or at least three gelling agents, at least one emollient, and at least 15% water by mass relative to the total mass.

### FIELD

The present disclosure relates generally to water in oil (W/O) emulsion compositions, which may be used in cosmetics. In particular, the present disclosure describes improved gelling systems for liquid W/O emulsions that allow the composition to contain a higher percentage of water while maintaining desirable characteristics such as a creamy and smooth texture, excellent coverage, and stability without separation of the underlying components and phases.

### BACKGROUND

The following discussion is merely provided to aid the reader in understanding the disclosure and is not admitted to describe or constitute prior art thereto.

Many of the currently available non-solid, water-containing cosmetics (skin products and color cosmetics) are designed to improve the look and feel of human skin. Typically, they contain water and humectants at above 10% and they are known in form of emulsions and gels with pigments at very low level (below 5%). Unfortunately for consumers, those of products may be very liquid and may not provide an aesthetic and even application and coverage. On another hand, the products containing a high level of pigments (above 5%) may appear to be sticky and very often unstable (separation and sedimentation occurs over time).

Accordingly, there remains a need for a gel based cosmetic which will provide long term moisturizing and cooling effects, exceptional coverage with minimal product deposit while at the same time is creamy, smooth, and stable. The present disclosure satisfies this need.

### SUMMARY

The present disclosure provides soft, creamy, smooth, and/or stable water in oil (W/O) emulsion compositions comprising a gelling system and an emollient.

In one aspect, the disclosure relates to a W/O emulsion composition, comprising, at least one gelling system, at least one emollient; and at least 15% water by mass relative to the total mass of the composition, wherein the at least one gelling system comprises at least one hydrophobic gelling agent and at least one hydrophilic gelling agent.

In some embodiments, the gelling system further comprises a hydrocarbon gelling agent. For example, in some embodiments, the hydrocarbon gelling agent is a hydrocarbon block copolymer. In some embodiments, the hydrocarbon gelling agent can be selected from ethylene/propylene/styrene copolymer, butylene/ethylene/styrene copolymer, or a mixture thereof. In some embodiments, the hydrocarbon gelling agent is present in a range of about 4% to about 20% by mass relative to the total mass of the composition.

In some embodiments, the hydrophilic gelling agent is selected from agar, agarose, carrageenan, gellan, alginic acid, and their mixtures thereof. In some embodiments, the hydrophilic gelling agent is present in a range of about 0.01% to about 1% by mass relative to the total mass of the composition.

In some embodiments, the hydrophobic gelling agent comprises a hydrophobic mineral gelling agent. In some embodiments, the hydrophobic mineral gelling agent is disteardimonium hectorite. In some embodiments, the hydrophobic mineral gelling agent is present in a range of about 1% to about 10% by mass relative to the total mass of the composition. For example, in some embodiments, the hydrophobic mineral gelling agent is about 6% by mass relative to the total mass of the composition.

In some embodiments, the emollient is a sterol derivative, such as Pandool®. In some embodiments, the emollient is present in a range of about 0.5% to about 10% by mass relative to the total mass of the composition. In some embodiments, the emollient is dimer dilinoleate phytosterol/higher alcohol ester.

In some embodiments, the amount of water is at least 25% by mass relative to the total mass of the composition.

In some embodiments, the W/O emulsion may further comprise at least one additional ingredient selected from a surfactant, a wax, a solvent, a texturing powder, a humectant, a pigment, and a film former.

In some embodiments, the W/O emulsion is incorporated into a lip composition.

In another aspect, the disclosure relates to methods of treating a keratinous surface comprising applying the composition of any one of the foregoing embodiments to a keratinous surface of a subject. In some embodiments, the keratinous surface may be the lips or skin of the subject (e.g., a human subject).

Also provided herein are compositions according to any one of the foregoing embodiments for treating a keratinous surface of a subject.

Also provided herein are compositions according to any one of the foregoing embodiments for use as a cosmetic.

Also provided herein are uses of a composition according to any one of the foregoing embodiments for treating a keratinous surface of a subject.

Also provided herein are uses of a composition according to any one of the foregoing embodiments as a cosmetic.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### DETAILED DESCRIPTION

The present disclosure provides W/O emulsion compositions comprising a gelling system, which may contain 2 or 3 or more gelling agents and a sterol based emollient. The disclosed W/O emulsions may be incorporated into creamy liquid cosmetics to provide a product that is soft, smooth, and stable and which can be easily and evenly applied to a user's keratinous surface, such as skin or lips.

Various patent application publications, patents, journal articles, and/or books may be referenced throughout the course of this disclosure. These references are not admitted to describe or constitute prior art, but are incorporated by reference herein in their entirety.

### I. Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, “about” means the recited quantity exactly and plus or minus 10%. For example, “about 10” should be understood to mean “10” and “9-11”.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the terms “individual,” “patient,” or “subject” can be an individual organism, a vertebrate, a mammal (e.g., a bovine, a canine, a feline, or an equine), or a human. These terms may be used interchangeably throughout the disclosure. In a preferred embodiment, the individual, patient, or subject is a human.

As used herein, “optional” or “optionally” means that the subsequently described event or circumstance may or may not occur, and that the description includes instances where said event or circumstance occurs and instances where it does not.

For the purpose of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B, and

C).

The description may use the terms “embodiment” or “embodiments,” which may each refer to one or more of the same or different embodiments.

Percentages are by mass (weight) of the composition or the particular phase being described, unless otherwise specified. All ratios are mass ratios, unless specifically stated otherwise.

A “liquid cosmetic” means that a composition is characterized by but not limited to viscosity below 150000 cps measured according to method described below. A “keratinous surface” as used herein means skin (including face), hair, including eyelashes and eyebrows, that might be treated with skin and/or makeup products (e.g., color cosmetics).

The term “oil phase” as used herein means a phase containing a hydrophobic carrier, typically oily ingredients. Suitable oils include, by way of example and not limitation, silicones oils (linear and cyclic), esters, ketons, glycol ethers vegetable and mineral oils, synthetic oils, paraffinic oils, hydrocarbons, aromatic solvents, including but not limited to those identified herein. The oils may be volatile or non-volatile, and are preferably in the form of pourable liquids at the room temperature (25° C.).

The oil phase may contain additional hydrophobic compounds that are easy to incorporate into the hydrophobic carriers. Such other applicable hydrophobic compounds can be in a liquid, solid and/or semi-solid form and they may, for example, include and are not limited to oil dissolvable waxes and film-formers, surfactants, emulsifiers, preservatives, thickeners (gellants), emollients, actives, vitamins, pigments, extracts, powders, hydrophobic solvents, and other useful ingredients.

“Volatile” means that the oil has a measurable vapor pressure of at least about 2 mm of mercury at 20° C.

The term “non-volatile” means that the oil has a vapor pressure of less than 2 mm of mercury at 20° C.

The term “hydrophobic” typically means that the substance, because of its nonpolar structure, is difficult to dissolve in water. The term “hydrophilic” on another hand means that the substance is dissolvable in water or other polar solvents due to its hydrogen bonding ability.

Some compounds may be characterized by having both hydrophobic and hydrophilic properties.

The term “stable composition” means that the formula stays uniform in its structure and does not separate to different phases.

The term “water phase” is understood to be a phase containing water as the dominant carrier. Typically, the water phase may contain additional substances, for example water dissolvable film-formers, surfactants, emulsifiers, thickeners (gellants), emollients, humectants, preservatives, actives, vitamins, extracts, powders, hydrophilic solvents, and other useful ingredients. Those compounds might be introduced to the composition in the liquid, solid and/or semi-solid form.

As will be understood by one skilled in the art, for any and all purposes, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having “1-3 gelling agents” refers to groups having 1, 2, or 3 gelling agents. Similarly, a group having “1-5 gelling agents” refers to groups having 1, 2, 3, 4, or 5 gelling agents, and so forth.

### II. Water in Oil Emulsion with Gelling System and Emollient

Non-solid water-containing cosmetics comprising a high levels of water (i.e., greater than 15% by weight) and humectants are known in the art. These known compositions are commonly in the form of gels or liquids. These cosmetics with a high level of water may contain pigments, but the compositions are typically unstable, meaning pigment sedimentation often occurs, and these products do not provide good coverage of the skin or lips. Moreover, known lip products containing around 20% water are not creamy and are often sticky, heavy feeling, and uncomfortable to wear.

The present disclosure provides water in oil (W/O) emulsion compositions comprising a gelling system and at least one emollient. Unlike prior W/O cosmetics and compositions, the disclosed compositions are characterized by smooth and creamy texture, which provides very good coverage using a single application, but at the same time the disclosed compositions are very light/weightless, making them comfortable to wear all day. It is easy to apply and remove the disclosed compositions without staining, and the disclosed compositions provide long term hydrating effects, which prevent the lips and skin from drying out during prolonged use. Additionally, the disclosed compositions do not separate over time, in contrast with many other products that separate of sediment under routine storage conditions.

Regardless of the high water level and concentration of texturizing powders and pigments, the disclosed compositions remain stable and possess great makeup retention. The disclosed W/O emulsions with an emollient are useful in applications for all kinds of color cosmetics as well as skin products.

The disclosed W/O emulsion compositions comprise a gelling system, which preferably comprises two or three gelling agents including a hydrocarbon gellant (e.g., Versagel®), a hydrophilic gelling agent (e.g., agars, such as Ina Agar CS-110), and a hydrophobic mineral gellant (e.g., Bentone gel)). The disclosed W/O emulsion compositions further comprise a sterol based emollient (such as Plandool®). The combination of more than one gelling agent and an emollient allows the disclosed compositions to retain a high level of water, such as more than 15%, while maintaining stability. As described in more detail below and in the Examples Section, this combination of components may provide surprising stability, hydration, and comfort, among numerous other beneficial physical and sensory characteristics.

In some embodiments, the disclosed W/O emulsion compositions comprises (i) a gelling system comprising at least one hydrocarbon gelling agent, at least one hydrophobic gelling agent, at least one hydrophilic gelling agent; (ii) an emollient; and at least 15% water by mass relative to the total mass of the composition.

In some embodiments, the hydrocarbon gelling agent is present in an amount between about 4% and about 20%. In some embodiments, the hydrocarbon gelling agent may comprise a hydrocarbon block copolymer, such ethylene/propylene/styrene copolymer and/or butylene/ethylene/styrene copolymer (Versagel®).

In some embodiments, the hydrophilic gelling agent is present in an amount between about 0.01% and about 1%. In some embodiments, the hydrophilic gelling agent may be an agar.

In some embodiments, the hydrophobic gelling agent is present in an amount between about 1% and about 10%. In some embodiments, the hydrophobic gelling agent may be bentone/di steardimonium hectorite.

In some embodiments, the emollient is present in an amount between about 0.5% and about 10%. In some embodiments, the emollient is bis-behenyl/phytosteryl dimer dilinoleate (Plandool®).

In some embodiments, water is present in an amount between about 15% and about 50%, or between about 15% and about 45%, or between about 15% and about 40%, or between about 15% and about 35%. In some embodiments, the amount of water may be greater than 15%, greater than 16%, greater than 17%, greater than 18%, greater than 19%, greater than 20%, greater than 21%, greater than 22%, greater than 23%, greater than 24%, greater than 25%, greater than 26%, greater than 27%, greater than 28%, greater than 29%, greater than 30%, greater than 31%, greater than 32%, greater than 33%, greater than 34%, or greater than 35%.

For example, in some embodiments, the disclosed W/O emulsion composition may comprise about 16% hydrocarbon gelling agent (e.g., ethylene/propylene/styrene copolymer and/or butylene/ethylene/styrene copolymer) about 0.35% hydrophilic gelling agent (e.g., agar), about 6% hydrophobic gelling agent (e.g., Benton/disteardimonium hectorite), about 4% emollient (e.g., bis-behenyl/phytosteryl dimer dilinoleate), and about 25% water.

As described in more detail below, the various embodiments of the disclosed W/O emulsion compositions comprising at least 15% water, a gellifying system, and an emollient provide excellent skin and lip coverage, exceptional hydration, and comfort during extended wear.

### A. Gelling

The W/O emulsion compositions of the present disclosure are characterized as comprising a gelling system comprising at least 2 and, preferably, at least 3 gelling agents. As used herein, “gelling agent” means an agent, contributing to modification of viscosity, thixotropic properties and stability of cosmetic compositions (e.g., reduces pigment sedimentation, syneresis). Thus, the disclosed gelling system may comprise, for example, at least one hydrophobic gelling agent and at least one hydrophilic gelling agent. In some embodiments, the gelling system may comprise a hydrocarbon gelling agent, a hydrophilic gelling agent, and a hydrophobic mineral gelling agent.

In some embodiments, the gelling system ranges from about 1.01% to about 33%, preferably from about 5% to about 25%, and most preferably from about 10% to about 22% by mass relative to the total mass of the composition. For example, the mass of the gelling system relative to the total mass of the composition may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, or about 33% or any value in between.

In some embodiments, the weight ratio of the hydrophobic gelling agent(s) and the hydrophilic gelling agent may be from about 1.5:0.005 to about 12.5:1, such as about 6.0:0.35.

In some embodiments, when incorporating at least one hydrocarbon gelling agent, such as a hydrophobic styrene copolymer gelling agent, the weight ratio of the hydrocarbon gelling agent and the hydrophobic gelling agent (e.g., hydrophobic mineral gelling agent; bentone/disteardimonium hectorite) is from about 4:1 to about 2:1, such as about 8:3. In some embodiments, the weight ratio of the hydrocarbon gelling agent (i.e., styrene copolymer in carrier) to the hydrophilic gelling agent (e.g., agar) to the hydrophobic gelling agent (e.g., mineral gellant in carrier) is from about 4:0.01:1 to about 2:0.1:1, respectively, such as about 16:0.35:6.

### i. Hydrocarbon Gelling Agent

For the purposes of the present disclosure, the gelling system of the disclosed W/O emulsion compositions may comprise at least one hydrocarbon gelling agent. Hydrocarbon gelling agents may include, but are not limited to, ethylene/propylene/styrene copolymers, butylene/ethylene/styrene co-polymers, or mixtures thereof. The presence of the hydrocarbon gelling agent results in a desirable viscosity, among other desirable properties, of the finished product.

In some embodiments, the at least one hydrophobic gelling agent may be a hydrocarbon block copolymer. In some embodiments, the hydrocarbon block copolymer comprises at least one styrene block and at least one block comprising units chosen from butadiene, ethylene, propylene, butylene, and isopropene, and hydrogenated hydrocarbon block copolymers and their mixtures thereof. Examples of suitable hydrocarbon-based block copolymers are described in U.S. Pat. No. 5,221,534, herein incorporated by reference. In some embodiments, the hydrocarbon based block copolymers may be soluble or dispersible in the oil phase. In some embodiments, the hydrocarbon based block copolymer is an amorphous block copolymer of styrene and olefin. In some embodiments, the hydrocarbon block copolymer may be a styrene copolymer gelling agent chosen from but not limited to block polymers comprising at least one block composed of styrene units or styrene derivatives. A copolymer comprising at least one styrene block may be a diblock or triblock copolymer or a multiblock copolymer, star or radial. Non-limiting examples of such copolymers include ethylene/propylene/styrene copolymer and butylene/ethylene/styrene as per description in U.S. Pat. No. 8,021,674 and for example as described in U.S. Pat. No. 6,433,068, all of which are herein incorporated by reference.

The term “polymers” is intended to mean both copolymers obtained from two types of monomers and those obtained from more than two types of monomers, such as, for example, terpolymers obtained from three types of monomers. The molecules of polymers may contain at least one hydrophilic unit and/or at least one hydrophobic unit.

One non-limiting exemplary commercially available styrene copolymer gelling agent useful in the disclosed compositions is Versagel ME 2000 (INCI name: Hydrogenated Polyisobutene (and) Ethylene/Propylene/Styrene Copolymer (and) Butylene/Ethylene/Styrene Copolymer) and other kinds of the gelling agents sold under the VERSAGEL® series available from Calumet Penreco. The applicable VERSAGEL® gellants contain styrene copolymers in a variety of carriers, such as for example mineral oil, isohexadecane, isododecane, hydrogenated polyisobutane, C12-15 alkyl benzoate, and isonolnyl isononanoate.

In some embodiments, the amount of the hydrocarbon gelling agent (i.e., the styrene copolymer in carrier) may range from about 4% to about 20%, for example, from about 6% to about 18%, or from about 8% to about 16% by mass relative to the total mass of the composition. For example, the amount of hydrocarbon gelling agent by mass relative to the total mass may be about 4%, about 5%, about 6%, about 7%, about 8%, about 9%, about 10%, about 11%, about 12%, about 13%, about 14%, about 15%, about 16%, about 17%, about 18%, about 19%, or about 20% or any value in between.

### ii. Hydrophobic Mineral Gelling Agents

For the purposes of the present disclosure, the gelling system of the disclosed W/O emulsion compositions may comprise at least one hydrophobic gelling agent. Hydrophobic gelling agents may include, but are not limited to, synthetic and/or natural viscosity increasing agents. The presence of the hydrophobic gellant may result in a desirable texture, hardness, and/or stability of the finished product.

In some embodiments, the at least one hydrophobic gelling agent may be a thickening hydrophobic gelling agent, such as, hydrophobic minerals and mixtures thereof.

Hydrophobic gelling agents are typically incorporated into the oil phase of the disclosed compositions, and the suitable hydrophobic gelling agents can be used in a form of solids or gels.

In some embodiments, the at least one hydrophobic gelling agent may comprise at least one hydrophobic mineral gellant. For the purposes of the disclosed compositions, useful hydrophobic mineral gellants can be chosen from, but are not limited to, organic modified clays and modified or unmodified hectorites and hydrophobic silicas, including fumed silicas. Thus, in some embodiments the hydrophobic mineral gelling agent may be selected from, for example, dimethyl distearyl ammonium hectorite, dimethyl distearyl ammonium bentonite, and dimethyl distearyl ammonium modified montmorillonite and others, as described and exemplified in U.S. Pat. Pub. No. 2007/0071703, which is hereby incorporated by reference. In some embodiments, the hydrophobic mineral gelling agents may be those in which a quaternary ammonium salt compound is added to a natural or synthetic smectite clay mineral, such as bentonite, by way of an ion exchange reaction. The choice of organic modified clay minerals is not particularly limited as long as it is cosmetically acceptable and may include, for example, dimethyl ammonium hectorite, benzyl dimethyl stearyl ammonium hectorite, and magnesium aluminum silicate treated with distearyl dimethyl ammonium chloride. In some embodiments, hydrophobic mineral gellants are selected, for example, from bentonites and organically modified hectorites pre-dispersed in organic solvents. A non-limiting example of commercially available bentonite is BENTONE GEL® series, including BENTONE GEL® ISD V (INCI: Isododecane, Disteardimonium Hectorite, Propylene Carbonate) available from Elementis Specialties. Another exemplary material is GARAMITE 7308XR (INCI: Quaternium-90 Sepiolite and Quaternium-90 Montmorillonite) available from Eckart. In some embodiments, an applicable hydrophobic mineral gelling agent comprises silica, in particular fumed silica and those treated with silanol groups. Such hydrophobic silicas are commercially available, for example, under the names of AEROSIL® by Degussa and CAB-O-SIL® available from Cabot.

Suitable mineral gelling agents may be utilized in a solid powder form or a gel, where the powder(s) are dispersed in a carrier, such as for example mineral oil, isohexadecane, isododecane, hydrogenated polyisobutane, C12-15 alkyl benzoate, and/or isonolnyl isononanoate.

In some embodiments, the hydrophobic gelling agents are present in an amount from about 1% to about 10%, for example, from about 2% to about 8% or from about 4% to about 6% by mass relative to the total mass of the composition. For example, the amount of hydrophobic gelling agent by mass relative to the total mass may be about 1%, about 2%, about 3%, about 4%, about 5%, about 6%, about 7%, about 8%, about 9%, or about 10% or any value in between.

### iii. Hydrophilic Gelling Agent

For the purposes of the present disclosure, the gelling system of the disclosed W/O emulsion compositions may comprise at least one hydrophilic gelling agent. Hydrophilic gelling agents may include, but are not limited to, thermoreversible polysaccharides, e.g., agar, agarose, carrageenan, and gellan. In some embodiments, the hydrophilic gelling agents can be incorporated into the formulations in the powder form or gels. The presence of the hydrophilic gelling agent allows for optimizing the desired texture and hardness.

In some embodiments, the at least one hydrophilic gelling agent is an agar as described in U.S. Pat. No. 8,933,134, which is hereby incorporated by reference. An agar useful in exemplary embodiments of the disclosed composition is insoluble in cold water, but may swell considerably, absorbing as much as twenty times its own weight of water. It dissolves readily in boiling water and sets to a firm gel at concentrations as low as 0.50%. Another useful example is powdered dry agar that is soluble in water and other solvents at temperatures between 95° and 100° C. A still further useful example is moistened agar flocculated by ethanol, 2-propanol or acetone, or salted out by high concentrations of electrolytes that is soluble in a variety of solvents at room temperature. In some embodiments, the preferred form of agar is the powder; however, agar can be also introduced to the composition as an agar gel, its preparation and characteristics are disclosed in U.S. Pat. Nos. 9,757,312 and 8,367,044, all of which are hereby incorporated by reference in their entirety. The agar used in connection with the disclosed composition may be of cosmetic as well as food grade, including the one available from Ina Food Industry Corporation.

The amount of the hydrophilic gelling agent to be used in the disclosed compositions ranges but is not limited to from about 0.01% to about 2%, for example, from about 0.1% to about 1%, or from about 0.2% to about 0.5% by mass relative to the total mass of the composition. In some embodiments, the hydrophilic gelling agent may be mixed with water to obtain a hydrophilic gel. The amount of the at least one hydrophilic gel may range from about 0.01% to about 1%, for example, from about 0.1% to about 0.9%, or from about 0.25% to about 0.5% by mass relative to the total mass of the composition. For example, the amount of hydrophilic gelling agent by mass relative to the total mass may be about 0.01%, about 0.02%, about 0.03%, about 0.04%, about 0.05%, about 0.06%, about 0.07%, about 0.08%, about 0.09%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.00% or any value in between.

### B. Emollient

In order to obtain the disclosed compositions containing water at high level (e.g., above 15%) and provide exceptional hydration, comfort, and/or a soft/satin effect, the disclosed W/O emulsion compositions may comprise at least one emollient. For the purposes of the present disclosure one of the suitable emollients are sterol derivatives.

Various emollients that may be incorporated into the disclosed compositions include sterol derivatives, petrolatum, lanolin, mineral oil, and dimethicone. Sterol derivatives are a particularly preferred emollient, but a person of skill in the art will understand that other similar emollients may be substituted.

The suitable sterol derivatives are phytosterols and cholesterols as those described in U.S. Pat. Pub. No. 2015/0202137. Examples of suitable phytosterols include dimer dilinoleate phytosterol/higher alcohol esters. The broad range of suitable dimer dilinoleate phytosterol/higher alcohol esters is known under the name of Plandool® available from Nippon Fine Chemical. Plandool® products are lanolin-like paste esters synthesized from plant-based materials. Pandool® compounds are characterized as having a high water holding capacity, high refractivity index, high pigment dispersing ability, high oxidation stability, excellent emolliency, excellent gloss, and thixotropic flow. Several exemplary Plandool® compounds are described in the table below.

TABLE 1
Types of Plandool

| Name | INCI Name |
|---|---|
| Plandool-H | PHYTOSTERYL/ISOSTEARYL/ |
| | CEIYL/STEARYL/BEHENYL |
| | DIMER DILINOLEATE |
| Plandool-S | PHYTOSTERYL/ISOSTEARYL/ |
| | CEIYL/STEARYL/BEHENYL |
| | DIMER DILINOLEATE |
| Plandool-G | BIS-BEHENYL/ISOSTEARYL/ |
| | PHYTOSTERYL DIMER |
| | DILINOLEYL DIMER |
| | DILINOLEATE |
| Plandool-GR | *BIS-BEHENYL/ISOSTEARYL/ |
| | PHYTOSTERYL DIMER |
| | DILINOLEYL DIMER |
| | DILINOLEATE, |
| | *GLYCERYL |
| | TRIHYDROGENATED |
| | ROSINATE |
| Plandool-PB | BIS-BEHENYL/ |
| | PHYTOSTERYL DIMER |
| | DILINOLEYL DIMER |
| | DILINOLEATE, TOCOPHEROL |

In some embodiments, the at least one emollient is present in an amount from about 0.5% to about 10%, for example, from about 2% to about 8% or from about 4% to about 6% by mass relative to the total mass of the composition. For example, the amount of emollient by mass relative to the total mass may be about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% or any value in between.

In some embodiments, particularly useful emollient is Bis-behenyl/phytosteryl dimer dilinoleate present in an amount from about 0.5% to about 10%, for example, from about 2% to about 8% or from about 4% to about 6% by mass relative to the total mass of the composition. For example, the amount of Bis-behenyl/phytosteryl dimer dilinoleate by mass relative to the total mass may be about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% or any value in between.

### C. Water

The disclosed W/O emulsions require water, and they may be unique in the ability to maintain stability and desirable texture (among other desirable characteristics) while incorporating a large percentage of water. In some embodiments, the disclosed W/O emulsions comprise about 15% water or greater than or equal to 15% water. For example, the amount of water may range from about 15% to about 50%, from about 15% to about 45%, from about 15% to about 40%, from about 15% to about 35%, from about 15% to about 30%, from about 15% to about 25%, from about 20% to about 50%, from about 20% to about 45%, from about 20% to about 40%, from about 20% to about 35%, from about 20% to about 30%, from about 20% to about 25%, from about 25% to about 50%, from about 25% to about 45%, from about 25% to about 40%, from about 25% to about 35%, from about 25% to about 30%, or any value in between by mass relative to the total mass of the composition. Indeed, in some embodiments, the amount of water in the W/O emulsion may be 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% or any value in between by mass relative to the total mass of the composition.

### D. Further Composition Components

The disclosed W/O emulsion compositions may further comprise additional components that render the composition suitable for use as a cosmetic or for other commercial purposes. The following components may be incorporated into the disclosed compositions comprising a gelling system, an emollient, and at least 15% water.

### i. Surfactant System

The disclosed compositions may comprise at least one surfactant system comprising, for example, at least one nonionic surfactant having a hydrophilic-lipophilic balance (HLB) value which equals or is less than 14. The HLB value is commonly associated with emulsifiers. In particular, the HLB value relates to the ratio of hydrophilic groups and lipophilic groups in emulsifiers, and relates to solubility of the emulsifiers. Typically, lower HLB emulsifiers are more soluble in lipophilic materials or oils and are more appropriate for use in W/O emulsions. On the other hand, higher HLB emulsifiers are more soluble in water or hydrophilic materials and are more suitable for oil in water (O/W) emulsions.

In some embodiments, the disclosed compositions may comprise, for example, a surfactant system comprising a blend of at least two nonionic surfactants having HLB values from 2-14. Examples of applicable nonionic surfactants may include polyglycerol alkyl ethers, ester-linked surfactants, polyoxyethylene alkyl ethers, polyglyceryl esters, glyceryl ethers, sorbitan derivatives, including sorbitan esters, polyethylene glycol derivatives of silicones, polyethylene glycol ethers, alkoxylated alcohols, and carbohydrates. By way of further example, suitable surfactants can include, but are not limited to, propylene glycol isostearate, glyceryl stearate, polygleceryl-3 diisostearate, sorbitan isostearate, oleth-2, glyceryl laurate, cetheth-2, methyl glucose sesquistearate, laureth-4, ceteryl glucoside, polysorbate 85, oleth-10 and ceteth-10.

In some embodiments, the disclosed compositions may comprise polyethylene glycol derivatives of dimethicone, specifically PEG-8 to PEG-12 dimethicone surfactants and those described in U.S. Pat. No. 7,842,725. In some embodiments, suitable but non-limiting examples of polyethylene glycol derivatives of dimethicone are PEG-12 dimethicone and PEG-10 dimethicone, both having HLB value of less than14. In some embodiments, the blend of dimethicone surfactants contains PEG-12 dimethicone and PEG-10 dimethicone at the mass ratio from about 5:1 to about 1:1, based on the total mass of the composition.

In some embodiments, the disclosed compositions may comprise at least two, at least 3, or at least 4 or more nonionic surfactants.

The amount of the nonionic surfactant may range, but is not limited to, from about 0.01% to about 4%, for example, from about 0.1% to about 3%, or from about 0.2% to about 2.5% by mass relative to the total mass of the composition. The amount of the glyceryl stearate surfactants may range, but is not limited to, from about 0.01% to about 4%, for example, from about 0.1% to about 3%, or from about 0.2% to about 2.5% by mass relative to the total mass of the composition.

The amount of the PEG-12 dimethicone may range, but is not limited to, from about 0.01% to about 4%, for example, from about 0.1% to about 3%, or from about 0.2% to about 2.5% by mass relative to the total mass of the composition.

The amount of the PEG-10 dimethicone may range, but is not limited to, from about 0.01% to about 4%, for example, from about 0.1% to about 3%, or from about 0.2% to about 2.5% by mass relative to the total mass of the composition.

### ii. Wax

In some embodiments, the disclosed compositions may comprise at least one wax. The at least one wax can be chosen from, but is not limited to, a solid or semisolid waxes at room temperature (25° C.). Specific examples include, but are not limited to, natural and synthetic waxes, for example: beeswax, candelilla wax, cotton wax, carnauba wax, bayberry wax, insect wax (wax secreted by Ericerus pela), spermaceti, montan wax, bran wax (rice wax), capok wax, Japan wax, lanolin acetate, liquid lanolin, sugar cane wax, esters of lanolin fatty acids and isopropyl alcohol, hexyl laurate, reduced lanolin, jojoba wax, hard lanolin, shellac wax, beeswax, microcrystalline wax, paraffin wax, POE lanolin alcohol ethers, POE lanolin alcohol acetates, POE cholesterol ethers, esters of lanolin fatty acids and polyethylene wax, synthetic wax, fatty acid glycerides, hydrogenated castor oil, petrolatum, and POE hydrogenated lanolin alcohol ethers.

The amount of the wax may range, but is not limited to, from about 2% to about 10%, for example, from about 2.5% to about 8%, or from about 3% to about 6% by mass relative to the total mass of the composition.

### iii. Solvents

In some embodiments, the disclosed compositions may comprise at least one solvent. The at least one solvent can be chosen from, but is not limited to, solvents having flash point below 50° C. and those which have flash point above 70° C., which includes volatile and non-volatile hydrocarbons, volatile and non-volatile silicones, alcohols, glycols, esters, vegetable oils, and synthetic oils. Some exemplary solvents include hydrocarbon and silicone based solvents, such as hydrogenated polyisobutane, isododecane, dimethicone, and semethicone. A commercially available suitable hydrocarbon solvent is PARLEM 4 (INCI name: Hydrogenated polyisobutane and tocopherol) available from NOF Corporation.

The amount of the solvent may range, but is not limited to, from about 0.01% to about 30%, for example, from about 0.1% to about 25%, or from about 1% to about 20% by mass relative to the total mass of the composition.

### iv. Texturizing Powders

In some embodiments, the disclosed compositions may comprise at least one texturing powder. The at least one texturizing powder can be chosen from, but is not limited to, mineral and organic fillers of any shape. For example, the texturizing powders can be, but are not limited to, a blend of silica, polymer microsphere, and amino acid. Examples include, but are not limited to, mica, silica, polyamide powders, starch, boron nitre, silicone resins microbeads, and elastomeric powders.

The amount of the texturizing powers may range, but is not limited to, from about 0.01% to about 30%, for example, from about 0.1% to about 25%, or from about 1% to about 20% by mass relative to the total mass of the composition.

### v. Humectants

Humectants, including glycerin, lecithin, and propylene glycol, draw water into the outer layer of skin. In some embodiments, the disclosed compositions may comprise one or more humectants selected from, but not limited to, hydrophilic humectants including polyol type humectants such as dipropylene glycol, polyglycerine, 1,3-butylene glycol, ethers, polyols, glycerine, glycerine polymers, and polyethylene glycol, as described in U.S. Pat. No. 5,977,188. In some embodiments, glycerin and glycerin polymers having up to 40 glycerin units are incorporated into the disclosed compositions as humectants.

The amount of the humectants ranges but is not limited from about 0.01% to about 5%, for example, from about 0.1% to about 3%, or from about 0.2% to about 1% by mass relative to the total mass of the composition.

### vi. Pigments

In some embodiments, the disclosed compositions may comprise at least one pigment chosen from, but not limited to, organic and inorganic treated pigments, and pigments providing special visual effects, for example. Particular examples of suitable pigments include, but are not limited to, iron oxide, red 7 lake, and gonioichromatic pigments.

The amount of the pigments may range, but is not limited to, from about 0.01% to about 30%, for example, from about 0.1% to about 25%, or from about 1% to about 20% by mass relative to the total mass of the composition.

### vii. Film Formers

In some embodiments, the disclosed compositions may comprise at least one film former chosen from, but not limited to, water soluble film formers and oil soluble firm formers, acrylic based film formers, silicone based film formers, for example the one described in US2009/001708.

“Film former” or “film forming polymer” as used herein means a polymer or resin that leaves a film on the substrate to which it is applied, for example, after a solvent accompanying the film former has evaporated, absorbed into an/or dissipated on the substrate.

The amount of the film formers may range, but is not limited to, from about 0.01% to about 30%, more preferably from about 0.1% to about 25%, most preferably from about 1% to about 20% by mass relative to the total mass of the composition.

The disclosed compositions may further include other components appropriate for its intended use such as organic pigments, preservatives, fillers, actives, sunscreens, additives, and/or additional solvent.

### III. Method of Making the Disclosed W/O Emulsions Comprising Emollient

The disclosed compositions may be prepared by mixing the emollient (e.g., Plandool®), a wax, and at least one solvent (e.g., parleam-4 and vegelight C912-LC) together at 90° C. until a uniform mixture is formed. Other hydrophobic components can be added and the mixture can be homogenized while still being heated at 90° C. All of the elements of the aqueous phase can be combined in a separate container and heated to 70-80° C. until a clear, uniform solution is obtained. The aqueous phase can then be added to the rest of the composition and blended for about 10 minutes at 90° C. or until a smooth and creamy texture is obtained. Appropriate compartments are filed up with the finished compositions.

### IV. Methods of Using the Disclosed W/O Emulsions Comprising Emollient

Disclosed herein are methods of using the disclosed W/O emulsion compositions comprising a gelling system, at least one emollient, and at least 15% water. Specifically, the compositions may be used to treat a keratinous surface, such as the lips or skin. The methods comprise applying the disclosed compositions to a keratinous surface in order to provide color, hydration, and/or smoothness to the keratinous surface. In other words, the present disclosure provides methods of using the disclosed compositions as a cosmetic. In some embodiments, the disclosed compositions may be applied in the form of a, lip gloss, liquid lip liner, or lip tint. In some embodiments, the described compositions can be but are not limited to lip products, foundations, blushes, eyeshadows, mascaras, eyeliners, skin care products, sun care products, etc. The disclosed compositions can be used in variety of applicable applicators and containers such as jars, pens, pencils, brushes, etc.

Also provided herein are the disclosed liquid W/O emulsion compositions comprising a gelling system, at least one emollient, and at least 15% water for use as a cosmetic or for application to a keratinous surface.

Also provided herein are uses of the disclosed liquid W/O emulsion compositions comprising a gelling system, at least one emollient, and at least 15% water for treatment of a keratinous surface or as a cosmetic.

### EXAMPLES

The following examples are given to illustrate the present disclosure. It should be understood, however, that the disclosure is not to be limited to the specific conditions or details described in these examples.

### Example 1—Preparing an Exemplary W/O Emulsion with Emollient

The following example describes on exemplary process for preparing an embodiment of the disclosed W/O emulsion composition comprising an emollient. The components that were used are listed in Table 2 below.

TABLE 2
Exemplary Composition Components

| | | | Composition 1 | Composition 2 |
|---|---|---|---|---|
| Phase | Trade Name | INCI Name | Weight (%) | Weight (%) |
| A | PARLEAM 4 | HYDROGENATED | 5.37 | 5.49 |
| | | POLYISOBUTENE, | | |
| | | TOCOPHEROL | | |
| A | PERFORMA V825 | SYNTHETIC WAX | 3.00 | 3.00 |
| | POLYMER | | | |
| A | PLANDOOL-PB | BIS-BEHENYL/ | 4.00 | 4.00 |
| | | PHYTOSTERYL DIMER | | |
| | | DILINOLEATE, | | |
| | | TOCOPHEROL | | |
| A | VEGELIGHT C912- | C9-12 ALKANE, | 5.00 | 5.00 |
| | LC | COCO- | | |
| | | CAPRYLATE/ | | |
| | | CAPRATE | | |
| B | BENTONE GEL | HYDROGENATED | 6.000 | 6.00 |
| | PARLEAM | POLYISOBUTENE, | | |
| | STANDARD | DISTEARDIMONIUM | | |
| | INTERMEDIATE | HECTORITE, | | |
| | | PROPYLENE | | |
| | | CARBONATE, | | |
| | | TOCOPHEROL | | |
| C | ACTICIRE MB | JOJOBA ESTERS, | 4.00 | 4.00 |
| | 5909 | HELIANTHUS | | |
| | | ANNULS SEED | | |
| | | CERA, ACACIA | | |
| | | DECURRENS | | |
| | | FLOWER CERA, | | |
| | | POLYGLYCERIN-3 | | |
| C | PERFORMALENE | POLYETHYLENE | 2.00 | 2.00 |
| | PL | | | |
| D | VERSAGEL ME | HYDROGENATED | 16.00 | 16.00 |
| | 2000 | POLYISOBUTENE, | | |
| | | ETHYLENE/PROPYLENE/ | | |
| | | STYRENE COPOLYMER, | | |
| | | BUTYLENE/ETHYLENE/ | | |
| | | STYRENE COPOLYMER, | | |
| | | BHT | | |
| E | PLUROL | POLYGLYCERYL-3 | 2.50 | 2.0 |
| | DIISOSTEARIQUE | DIISOSTEARATE | | |
| | CG#5793 | | | |
| E | | | | 0.5 |
| E | | | | 0.01 |
| F | SW65U | CI 77891, | 7.200 | 7.20 |
| | | SYNTHETIC | | |
| | | WAX, ISOPROPYL | | |
| | | TITANIUM | | |
| | | TRIISOSTEARATE | | |
| F | SW40R7C | SYNTHETIC | 2.04 | 2.00 |
| | | WAX, CI 15850, | | |
| | | ISOPROPYL | | |
| | | TITANIUM | | |
| | | TRIISOSTEARATE | | |
| F | SW50EY | SYNTHETIC | 7.06 | 7.00 |
| | | WAX, CI 77492, | | |
| | | ISOPROPYL | | |
| | | TITANIUM | | |
| | | TRIISOSTEARATE | | |
| F | SW55EB | CI 77499, | 0.62 | 0.59 |
| | | SYNTHETIC | | |
| | | WAX, ISOPROPYL | | |
| | | TITANIUM | | |
| | | TRIISOSTEARATE | | |
| G | SATINIER M5 | SILICA | 2.00 | 2.00 |
| G | PLASTIC | HDI/TRIMETHYLOL | 2.00 | 2.00 |
| | POWDER D-400 | HEXYLLACTONE | | |
| | | CROSSPOLYMER, | | |
| | | SILICA | | |
| G | AMIHOPE LL | LAUROYL | 3.00 | 3.00 |
| | | LYSINE | | |
| G | CASHMIR B-3 | MICA, SILICA | 1.880 | 1.88 |
| H | DEIONIZED | AQUA | 25.00 | 25.00 |
| | WATER | | | |
| H | GLYCERINE NAT | GLYCERIN | 0.50 | 0.50 |
| | 99.7% ACIDCHEM | | | |
| H | INA AGAR CS-110 | AGAR | 0.35 | 0.35 |
| H | LEXGARD E | ETHYLHEXYLGLYCERIN | 0.38 | 0.38 |
| H | BIO-HYARO 12 | SODIUM | 0.10 | 0.10 |
| | | HYALURONATE | | |

Compounds A were combined in a main vessel and then heated at 90° C. until a uniform mixture was formed. Next, compounds B, C, D, E, F and G were added and homogenized for 2 minutes using Robomics (supplied by Tokushu Kika) at the speed of 4000 RPM, while still being heated at 90° C.

In the meantime, all elements of the water phase (H) were combined in a small container and heated at 70-80° C. until a clear and uniform blend was obtained.

Next, the water phase was added to the mixture in the main kettle and blended together for another 10 minutes at 90° C. until a smooth and creamy texture was obtained.

### Example 2—Determination of Qualities of W/O Emulsion with Emollient

The following example was used to determine the effect of each component of the gelling system and the emollient on the physical properties of the resulting composition. Accordingly, one composition was prepared as an exemplary W/O emulsion containing three gelling agents (i.e., a hydrophobic gelling agent, a hydrophilic gelling agent, and a hydrocarbon gelling agent) with emollient, and four controls were prepared, each with one of the components of a gelling agent or emollient missing. The components and concentrations of components for each of these compositions is disclosed in Table 3 below.

TABLE 3
Components of Exemplary and Control Compositions

| | | Exemplary | | | | |
|---|---|---|---|---|---|---|
| | | W/O | | | | |
| | | emulsion/ | Control-1 | Control-2 | | Control-4 |
| | | Emollient | (no | (no | Control-3 | (no |
| Trade name | INCI name | Composition | versagel) | Bentone) | (no Agar) | Plandool) |
| DEIONIZED | WATER | 25.000000 | 25.000000 | 25.000000 | 25.350000 | 25.000000 |
| WATER | | | | | | |
| VERSAGEL ME | HYDROGENATED | 16.000000 | 0.000000 | 16.000000 | 16.000000 | 16.000000 |
| 2000 | POLYISOBUTENE & | | | | | |
| | ETHYLENE/ PROPYLENE/ | | | | | |
| | STYRENE COPOLYMER & | | | | | |
| | BUTYLENE/ ETHYLENE/ | | | | | |
| | STYRENE COPOLYMER & | | | | | |
| | BHT | | | | | |
| SW65U | TITANIUM DIOXIDE & | 7.200000 | 7.200000 | 7.200000 | 7.200000 | 7.200000 |
| | SYNTHETIC WAX & | | | | | |
| | ISOPROPYL TITANIUM | | | | | |
| | TRIISOSTEARATE | | | | | |
| SW50EY | SYNTHETIC WAX & IRON | 7.060000 | 7.060000 | 7.060000 | 7.060000 | 7.060000 |
| | OXIDES & ISOPROPYL | | | | | |
| | TITANIUM | | | | | |
| | TRIISOSTEARATE | | | | | |
| BENTONE GEL | HYDROGENATED | 6.000000 | 6.000000 | 0.000000 | 6.000000 | 6.000000 |
| PARLEAM | POLYISOBUTENE & | | | | | |
| STANDARD | DISTEARDIMONIUM | | | | | |
| INTERMEDIATE | HECTORITE & | | | | | |
| | PROPYLENE | | | | | |
| | CARBONATE & | | | | | |
| | TOCOPHEROL | | | | | |
| PARLEAM 4 | HYDROGENATED | 5.370000 | 21.370000 | 11.370000 | 5.370000 | 9.370000 |
| | POLYISOBUTENE & | | | | | |
| | TOCOPHEROL | | | | | |
| VEGELIGHT | C9-12 ALKANE & COCO- | 5.000000 | 5.000000 | 5.000000 | 5.000000 | 5.000000 |
| C912-LC | CAPRYLATE/CAPRATE | | | | | |
| PLANDOOL-PB | BIS- | 4.000000 | 4.000000 | 4.000000 | 4.000000 | 0.000000 |
| | BEHENYL/PHYTOSTERYL | | | | | |
| | DIMER DILINOLEATE & | | | | | |
| | TOCOPHEROL | | | | | |
| ACTICIRE MB | JOJOBA ESTERS & | 4.000000 | 4.000000 | 4.000000 | 4.000000 | 4.000000 |
| 5909 | HELIANTHUS ANNUUS | | | | | |
| | (SUNFLOWER) SEED | | | | | |
| | WAX & ACACIA | | | | | |
| | DECURRENS FLOWER | | | | | |
| | WAX & POLYGLYCERIN- | | | | | |
| | 3 | | | | | |
| PERFORMA V825 | SYNTHETIC WAX | 3.000000 | 3.000000 | 3.000000 | 3.000000 | 3.000000 |
| POLYMER | | | | | | |
| AMIHOPE LL | LAUROYL LYSINE | 3.000000 | 3.000000 | 3.000000 | 3.000000 | 3.000000 |
| PLUROL | POLYGLYCERYL-3 | 2.500000 | 2.500000 | 2.500000 | 2.500000 | 2.500000 |
| DIISOSTEARIQUE | DIISOSTEARATE | | | | | |
| CG #5793 | | | | | | |
| SW40R7C | SYNTHETIC WAX & RED | 2.040000 | 2.040000 | 2.040000 | 2.040000 | 2.040000 |
| | 7 LAKE & ISOPROPYL | | | | | |
| | TITANIUM | | | | | |
| | TRIISOSTEARATE | | | | | |
| SATINIER M5 | SILICA | 2.000000 | 2.000000 | 2.000000 | 2.000000 | 2.000000 |
| PLASTIC | HDI/TRIMETHYLOL | 2.000000 | 2.000000 | 2.000000 | 2.000000 | 2.000000 |
| POWDER D-400 | HEXYLLACTONE | | | | | |
| | CROSSPOLYMER & | | | | | |
| | SILICA | | | | | |
| PERFORMALENE | POLYETHYLENE | 2.000000 | 2.000000 | 2.000000 | 2.000000 | 2.000000 |
| PL | | | | | | |
| CASHMIRB-3 | MICA & SILICA | 1.880000 | 1.880000 | 1.880000 | 1.880000 | 1.880000 |
| SW55EB | IRON OXIDES & | 0.620000 | 0.620000 | 0.620000 | 0.620000 | 0.620000 |
| | SYNTHETIC WAX & | | | | | |
| | ISOPROPYL TITANIUM | | | | | |
| | TRIISOSTEARATE | | | | | |
| GLYCERINE NAT | GLYCERIN | 0.500000 | 0.500000 | 0.500000 | 0.500000 | 0.500000 |
| 99.7% ACIDCHEM | | | | | | |
| LEXGARD E | ETHYLHEXYLGLYCERIN | 0.380000 | 0.380000 | 0.380000 | 0.380000 | 0.380000 |
| INA AGAR CS-110 | AGAR | 0.350000 | 0.350000 | 0.350000 | 0.000000 | 0.350000 |
| BIO-HYARO 12 | SODIUM HYALURONATE | 0.100000 | 0.100000 | 0.100000 | 0.100000 | 0.100000 |

Once the exemplary composition and the Controls were prepared, they were subjected to a battery of tests in order to assess their physical properties, including texture, viscosity, emulsion stability, and storage stability.

Texture was analyzed by two (2) users of liquid lip cosmetics after immediate application of the exemplary composition and the controls. The samples were assessed for: duration (i.e., how long lasting), lightness (weigh), refreshing and cooling sensation, smoothness, creaminess, coverage, and comfort after application.

Viscosity was measured using a Brookfield viscometer, model RV, using spindle T-D at speed of 12 RPM at 25° C./35% RM.

For assessing emulsion stability, the samples were visually inspected for separation immediately after making them.

For assessing storage stability, the samples were visually inspected for separation after being exposed for two (2) weeks at temperatures as follows: −5° C., 0° C., 25° C., 37° C., 45° C. and 37° C./98% RH (relative humidity). The results for each of these assessments are shown in Table 3 below.

TABLE 4
Comparison of Physical Properties of Various Compositions

| | Exemplary | | | | Control 4 |
|---|---|---|---|---|---|
| | Versagel + | Control 2 | Control 3 | Control 1 | Agar + |
| | Agar + | Versagel + | Versagel + | Agar + | Benton + |
| Para- | Benton + | Agar + | Benton + | Benton + | Versagel (no |
| meters | Plandool | Plandool | Plandool | Plandool | Plandool) |
| Texture | 3 | 2 | 2 | 2 | 2 |
| Viscosity | ◯ | X | X | X | X |
| Emulsion | ◯ | Δ | Δ | Δ | Δ |
| Stability | ◯ | X | X | X | X |

Texture Scale:
3: Very Good: texture has all these benefits: light weight, long lasting, refreshing, cooling sensation with soft, smooth, creamy, good and comfortable coverage
2: Acceptable: texture does not have all of the benefits listed in 3
1: Not Acceptable: texture doesn't have any of the benefits listed in 3
Viscosity:
◯ Very Good: viscosity with the range of 64000 cp to 136000 cp
Δ Acceptable: 40000 cps-150000 cps
X Not Acceptable: viscosity outside of acceptable range
Emulsion:
◯ Very Good: no separation
Δ Acceptable: slight separation
X Not Acceptable: observable separation
Stability:
◯ Very Good: no separation at −5° C., 0° C., 25° C., 37° C., 45° C. and 98% humidity for two weeks
Δ Acceptable: separation at most extreme temperatures
X Not Acceptable: separation observed under conditions listed above

As can be seen in Table 3 above, the W/O emulsion characterized with the most desirable texture, as well as acceptable viscosity and stability was the triple gel with emollient. These results suggest that three gelling agents and an emollient are needed to obtain the best possible results.

### Example 3—Comparison of W/O Emulsion with Emollient to Other Liquid Lip Products

In order to confirm the superior qualities of the disclosed triple gel with emollient compositions (e.g., ease of applicability and removal, coverage, weight, comfort, stickiness, finish, smell, etc.), the exemplary triple gel with emollient composition from Example 2 was compared against several commercially available products with high water content (i.e., above 15% for all except Comparison Product 3, as shown below).

Comparison Product 1 is a vinyl cream stain comprising water, octyldodecanol, trimethylsiloxyphenyl dimethicone, ethylcellulose, synthetic wax, cetyl alcohol, alcohol, polybutene, glyceryl stearate, behenyl alcohol, steareth-20, phenoxyethanol, sodium laurylsulfate, glyceryl stearate citrate, disodium ethylene dicocamide PEG-15 disulfate, hydroxypropyl guar, calcium aluminum borosilicate, synthetic fluorphlogopite, stearyl alcohol, myristyl alcohol, magnesium silicate, calcium sodium borosilicate, aluminum hydroxide, sodium saccharin, alumina, benzyl alcohol, silica, tin oxide, anise alcohol, fragrance, and pigments.

Comparison Product 2 is a lipstick comprising water, octyldodecanol, cyclopentasiloxane, hydrogenated polyisobutene, pentaerythrityl tetraisostearate, ethylcellulose, glycerin, alcohol, polyglyceryl-2 triisostearate, isododecane, polyglyceryl-6 polyricinoleate, pentylene glycol, polyglyceryl-2 isostearate, phenoxyethanol, disteardimonium hectorite, lecithin, butylene/ethylene/styrene copolymer, ethylene/propylene/styrene copolymer, trimethylolpropane triisostearate, Chondrus crispus (carrageenan), sodium chloride, BHT, parfum (fragrance), diisostearyl malate, polyglyceryl-3 polyricinoleate, stevioside, synthetic fluorphlogopite, benzyl salicylate, linalool, tocopherol, amyl cinnamal, propyl gallate, and pigments.

Comparison Product 3 is a lip tint comprising water, octyldodecanol, trimethylsiloxysilicate, cyclopentasiloxane, alcohol, sorbitan sesquioleate, polyglyceryl-2 triisostearate, dimer dilinoleyl dimer dilinoleate, caprylic/capric triglycerides, propylene glycol, polysorbate 80, ethylcellulose, polyacrylamide, C13-14 isoparaffin, laureth-7, 1,2-hexanediol, phenoxyethanol, Rosa canina fruit oil, Persea gratissima (avocado) oil, Prunus armeniaca (apricot) kernel oil, Macadamia ternifolia seed oil, tocopheryl acetate, fragrance, and pigment.

Comparison Product 4 is a lipstick comprising water, glycerin, butylene glycol, ethylhexyl stearate, alcohol, hydroxyethyl acrylate/sodium acryloyldimethyl taurate copolymer, vinyl dimethicone/methicone silsesquioxane crosspolymer, squalane, Phyllostachis bambusoides extract, phenoxyethanol, polyglyceryl-2 triisostearate, PEG-60 hydrogenated castor oil, lecithin, trimethylolpropane triisostearate, polysorbate 60, vanillin, parfum (fragrance), sodium citrate, sorbitan isostearate, stevioside, PEG-7 glyceryl cocoate, citric acid, polyquaternium-7, propyl gallate, benzyl alcohol, BHT, methylsilanol tri-PEG-8 glyceryl cocoate, and pigments.

The exemplary W/O emulsion with emollient composition and the comparison products were compared by application of four (4) regular users of liquid lip cosmetics, ranging in age from 24-55. The subjects applied the exemplary composition and each of comparison products and evaluated them for variety of parameters as listed in Table 4 below. Depending on the type of question, some assessments were done immediately after application and others after one (1) hour of wearing each of the products. The assessment of each of the products was conducted on different days, one product per day randomly chosen.

TABLE 5
Sensory Comparison of Exemplary and Competitor Compositions

| | Exemplary | | | | |
|---|---|---|---|---|---|
| | formulation | Comparison | Comparison | Comparison | Comparison |
| | with Emollient | Product 1 | Product 2 | Product 3 | Product 4 |
| % Water by KF | 25.00% | 20.57% | 20.68% | 9.08% | 61.69% |
| Coverage | Yes (Very good) | Yes | Yes | Yes | No |
| Easy application | Yes (very) | Yes | Yes | No | yes |
| Easy to remove | Yes | No (it stains) | Yes | Yes | No (it stains) |
| Smooth even | Yes | No | Yes | Yes | Yes |
| application | | | | | |
| Does not feel | Yes | Yes | No | Yes | Yes |
| tight on the lip | | | | | |
| Non-sticky | Yes | No | No | Yes | Yes |
| Light weight | Yes (very) | No | No (heavy) | Yes | Yes |
| Comfortable | Yes | No | No | No | No |
| Comfortable to | Yes | No | No | No | No |
| wear all day | | | | | |
| Does not dry out | Yes | Yes | Yes | Yes | Yes |
| lips | | | | | |
| Does not flake | Yes | Yes | Yes | Yes | Yes |
| or peel | | | | | |
| Satin finish | Yes | Yes | Yes | Yes | No |
| Smell | No | Alcohol Smell | Alcohol Smell | Alcohol/rancid | No |
| | | | | Smell | |

As shown in the table above, the triple gel with emollient performed better on every sensory assessment compared to comparison products. In particular, the disclosed triple gel with emollient compositions are easy to remove (i.e., do not stain), are not sticky or heavy, do not smell, and are comfortable to wear all day.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

In addition, where features or aspects of the disclosure are described in terms of Markush groups, those skilled in the art will recognize that the disclosure is also thereby described in terms of any individual member or subgroup of members of the Markush group.

1. A W/O emulsion composition, comprising, at least one gelling system, at least one emollient; and at least 15% water by mass relative to a total mass of the composition, wherein the at least one gelling system comprises at least one hydrophobic gelling agent and at least one hydrophilic gelling agent.

2. The W/O emulsion composition of claim 1, wherein the gelling system further comprises a hydrocarbon gelling agent.

3. The W/O emulsion composition of claim 2, wherein the hydrocarbon gelling agent is a hydrocarbon block copolymer.

4. The W/O emulsion composition of claim 2, wherein the hydrocarbon gelling agent is selected from ethylene/propylene/styrene copolymer, butylene/ethylene/styrene copolymer, or a mixture thereof.

5. The W/O emulsion composition of claim 2, wherein the hydrocarbon gelling agent is present in a range of about 4% to about 20% by mass relative to the total mass of the composition.

6. The W/O emulsion composition of claim 1, wherein the hydrophilic gelling agent is selected from agar, agarose, carrageenan, gellan, alginic acid, and their mixtures thereof.

7. The W/O emulsion composition of claim 1, wherein the hydrophilic gelling agent is present in a range of about 0.01% to about 1% by mass relative to the total mass of the composition.

8. The W/O emulsion composition of claim 1, wherein the hydrophobic gelling agent comprises a hydrophobic mineral gelling agent.

9. The W/O emulsion composition of claim 1, wherein the hydrophobic gelling agent is disteardimonium hectorite.

10. The W/O emulsion composition of claim 1, wherein the hydrophobic gelling agent is present in a range of about 1% to about 10% by mass relative to the total mass of the composition.

11. The W/O emulsion composition of claim 13, wherein the hydrophobic gelling agent is about 6% by mass relative to the total mass of the composition.

12. The W/O emulsion composition of claim 1, wherein the emollient is a sterol derivative.

13. The W/O emulsion composition of claim 1, wherein the emollient is present in a range of about 0.5% to about 10% by mass relative to the total mass of the composition.

14. The W/O emulsion composition of claim 1, wherein the emollient is dimer dilinoleate phytosterol/higher alcohol ester.

15. The W/O emulsion composition of claim 1, wherein the amount of water is at least 25% by mass relative to the total mass of the composition.

16. The W/O emulsion composition of claim 1 further comprising at least one surfactant, wax, solvent, texturing powder, humectant, pigment, or film former.

17. The W/O emulsion composition of claim 1, wherein the W/O emulsion is incorporated into a lip composition.

18. A method of treating a keratinous surface comprising applying the composition of claim 1 to a keratinous surface of a subject.

19. The method of claim 18, wherein the keratinous surface is lips or skin of the subject.

20. A composition according to claim 1 for treating a keratinous surface of a subject.

21. A composition according to claim 1 for use as a cosmetic.

22. Use of a composition according to claim 1 for treating a keratinous surface of a subject.

23. Use of a composition according to claim 1 as a cosmetic.
